(19) United States
(12) Plant Patent Application Publication
(10) Pub. No.: US 2024/0172572 P1
(43) Pub. Date: May 23, 2024

(54) PHILODENDRON PLANT NAMED 'NATPHI01'

(50) ;

(71) Applicant: Benjamin William Gregory, North Sydney (AU)

(72) Inventor: Benjamin William Gregory, North Sydney (AU)

(21) Appl. No.: 18/445,619

(22) Filed: Nov. 22, 2023

(30) Foreign Application Priority Data
Nov. 22, 2022 (QZ) 2022/2616

(51) Int. Cl. A01H 6/00

(52) U.S. Cl. PLT/381

(57) ABSTRACT

‘NATPH101’ is a distinctive Philodendron plant which is characterized by the combination of a broad spreading to upright growth habit with uniquely colored laminas borne on dark greyed-red petioles that fade with age to dark green with heavy greyed-red to greyed-orange striping, a dark green mature adaxial foliar surface with a light pink general coloration adjacent to the lateral venation, a yellow-green mature adaxial foliar surface with greyed-purple veins and a greyed-orange coloration adjacent to the venation, as well as the uniformity and stability of these characteristics from generation to generation.

Latin name of the genus and species

The Latin name of the genus and species of the novel variety disclosed herein is Philodendron erubescens.

Variety denomination

The inventive variety of Philodendron disclosed herein has been given the variety denomination ‘NATPH101’.

### BACKGROUND OF THE INVENTION

Parentage: ‘NATPH101’ is a spontaneous partial-plant mutation which was discovered in 2018, in Somerset, The United Kingdom, growing on a cultivated Philodendron erubescens ‘Birkin’ plant (not patented) in a greenhouse owned by the inventor. The mutation was first noted for its prominent pink veins and general pink coloration of the abaxial leaf surface.

Asexual Reproduction: Asexual reproduction of the new cultivar ‘NATPH101’, by way of meristematic tissue culture, was first initiated in 2021 in Somerset, The United Kingdom. Through ten subsequent generations, the unique features of this cultivar have proven to be stable and true to type.

### SUMMARY OF THE INVENTION

The cultivar ‘NATPH101’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘NATPH101’. These characteristics in combination distinguish ‘NATPH101’ as a new and distinct Philodendron cultivar:
1. Philodendron ‘NATPH101’ exhibits a broad spreading to upright growth habit with uniquely colored laminas borne on dark greyed-red petioles that fade with age to dark green with heavy greyed-red to greyed-orange striping; and
2. Philodendron ‘NATPH101’ exhibits a dark green mature adaxial foliar surface with a light pink general coloration adjacent to the lateral venation; and
3. Philodendron ‘NATPH101’ exhibits a yellow-green mature adaxial foliar surface, with greyed-purple veins, and a greyed-orange coloration adjacent to the venation.

### BRIEF DESCRIPTION OF THE FIGURES

FIG. 1 illustrates, as nearly true as it is reasonably possible to make the same in color photographs of this type, an exemplary plant of ‘NATPH101’ grown in a commercial greenhouse in Somerset, The United Kingdom. This plant is approximately 12 months old, in a 9 cm container.

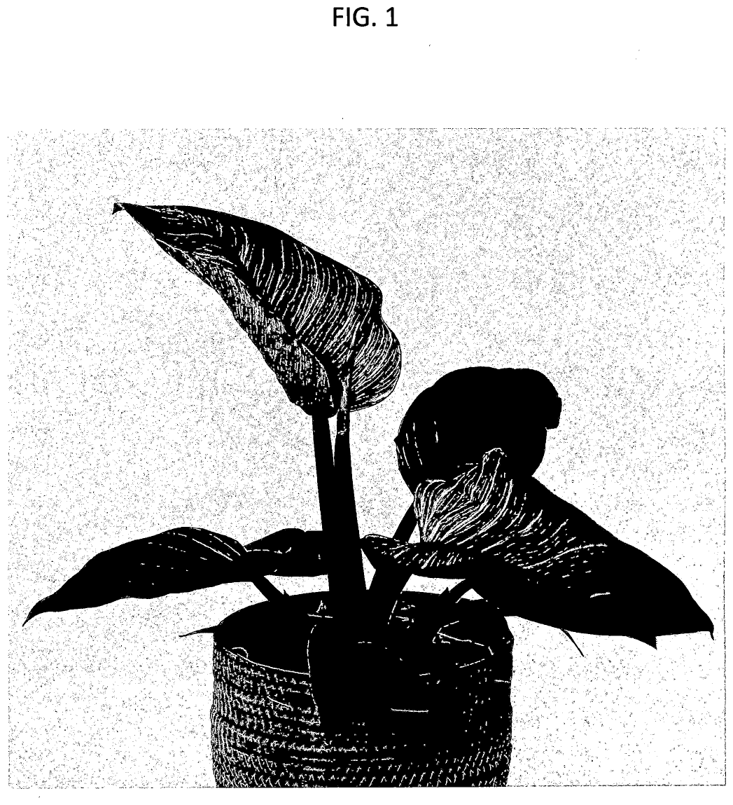

FIG. 2 illustrates, as nearly true as it is reasonably possible to make the same in color photographs of this type, the typical mature foliage of ‘NATPH101’.

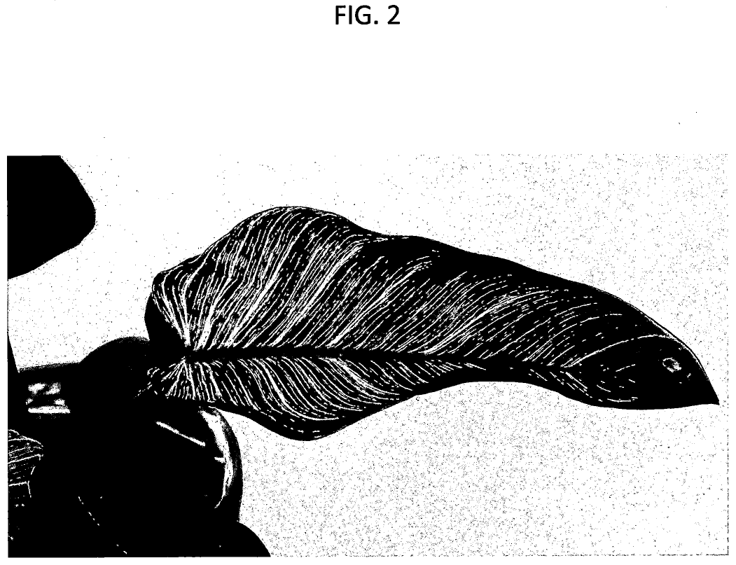

### BOTANICAL DESCRIPTION OF THE PLANT

The following observations and measurements, made in October of 2023, describe averages from a 12-month-old ‘NATPH101’ plant grown in 17 cm nursery containers at a greenhouse in Somerset. Plants were produced using conventional greenhouse production protocols for Philodendron sp. which consisted of growing plants under shade, regular overhead irrigation, supplemental fertilizer at rate of 1.5 EC (electroconductivity). No chemical pest treatments or photoperiodic treatments were utilized.

Those skilled in the art will appreciate that certain characteristics will vary with older or, conversely, with younger plants. ‘NATPH101’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, Sixth Edition.

A botanical description of ‘NATPH101’ and a comparison with the parent plant and most similar commercial cultivar are provided below.
Plant description:
   Growth habit.—Broad spreading to upright herbaceous perennial with foliage growth from basal shoots.
   Plant profile shape.—Globular to flattened globular.
   Height from soil level to top of foliar plane.—32.0 cm.
   Plant spread.—56.0 cm.
   Number of basal shoots per plant.—1.
   Growth rate.—Moderately fast.
   Plant vigor.—Moderately vigorous.
   Propagation.—Type — Asexual propagation is accomplished by way of stem and leaf cuttings which root well without rooting compound or hormone. Time to initiate rooting — Approximately 10 to 21 days at 22 degrees Celsius. Time to produce a rooted cutting — Approximately 6 to 9 months, at an ambient temperature range of 22 degrees Celsius, are needed to produce a marketable finished plant from a rooted cutting.
   Disease and pest resistance or susceptibility.—Neither susceptibility nor resistance to pests and diseases common to either Philodendron have been observed.
   Environmental tolerances.—Adapt to temperatures as loW as 5 degrees Celsius and at least as high as 40 degrees Celsius; moderate to high tolerance to rain; low to moderate tolerance to wind.
Roots:
   Density.—Not very dense.
   Texture.—Fleshy, non-fibrous.
   Color.—Greyed-orange, RHS 164A.
Foliage:
   Quantity.—7 leaves per shoot.
   Arrangement.—Alternate.
   Attachment.—Petiolate.
   Division.—Simple.
   Lamina.—Shape — Ovate. Length — 27.6cm, excluding the petiole. Width —15.2 cm. Apex — Apiculate. Base — Cordate to reniform; basal lobes are free. Aspect — Slightly to moderately concave. Margins — Entire; slightly repand; slightly coarsely undulated; minutely revolute. Texture and luster, adaxial surface — Smooth, glabrous and slightly glossy. Texture and luster, abaxial surface — Smooth, glabrous and slightly glossy. Color — Juvenile foliage, adaxial surface — Yellow-green, nearest to RHS 146A; areas adjacent to lateral veins are greyed-orange, nearest to a mixture of RHS N170C and N170D; narrowly margined greyed-purple, nearest to RHS 183A. Juvenile foliage, abaxial surface — Nearest to a mixture of yellow-green and greyed-green, RHS 146A and 189A; lateral veins are red-purple, nearest to RHS 61A, and areas adjacent to lateral veins are greyed-orange, nearest to RHS 174D; narrowly margined greyed-purple, nearest to RHS 185A. Mature foliage, adaxial surface — Green, nearest to RHS 139A yet considerably darker; areas adjacent to lateral veins are orange-white, nearest to RHS 159C; narrowly margined greyed-purple, nearest to RHS 187C. Mature foliage, abaxial surface — Yellow-green, nearest to a mixture of RHS 146A and 147B; areas adjacent to lateral veins are greyed-orange, nearest to RHS 174D; narrowly margined greyed-purple, nearest to RHS 183A. Mature venation — Pattern — Pinnate. Color, adaxial surfaces — The main veins and lateral veins are yellow-green, nearest to RHS 146A. Color, abaxial surface — The main veins and lateral veins are greyed-purple, nearest to RHS 183B.
   Petiole.—Attitude — Upright and outward. Strength — Moderately strong. Aspect — Subterete. Length — 16.7 cm. Height — 1.4 cm. Width — 1.0 cm. Texture and luster, adaxial surface — Smooth, glabrous and slightly glossy. Texture and luster, abaxial surface — Smooth, glabrous and slightly glossy. Juvenile color, adaxial surface — Greyed-red, nearest to RHS 181B. Juvenile color, abaxial surface — Greyed-red, nearest to RHS 181B. Mature color, adaxial surface — Nearest to a mixture of yellow-green and greyed-green, RHS 147A and N189A; heavily longitudinally striped with greyed-red, nearest to RHS 181B. Mature color, abaxial surface — Yellow-green, nearest to RHS 147A; heavily longitudinally striped with greyed-orange, nearest to RHS 176C. Petiole Wings — Position — Proximal end of the petiole. Length — 9.1 cm. Width — 0.7 cm. Color, adaxial surface — Greyed-purple, nearest to RHS 183D, and heavily longitudinally striped greyed-purple, nearest to N186C. Color, abaxial surface — Greyed-purple, nearest to RHS 183A, and heavily longitudinally striped with brown, nearest to 200B.
   Stipule.—Position — A single, non-persistent stipule is present at the base of each leaf, covering immature leaves before unfolding and senescing once the leaves unfold. Shape — Lanceolate. Aspect — Concave. Apex — Narrowly praemorse. Base — Attenuate. Margins — Entire. Length — 16.0 cm. Width — 4.2 cm. Texture, adaxial surface — Smooth; glabrous. Texture, abaxial surface — Smooth; glabrous. Color, adaxial surface — Greyed-purple, nearest to RHS 184B; and longitudinally striped darker, nearest to RHS 187B. Color, abaxial surface — Greyed-purple, nearest to a mixture of RHS 187B and 187C.
Inflorescence To date, ‘NATPH101’ has not flowered.
Comparisons with the parent plant and closest known commercial comparator:

Plants of the new cultivar ‘NATPH101’ differ from the seed parent, Philodendron ‘Birkin’ (not patented), which is also the most similar cultivar known to the inventor, by the characteristics described in Table 1 below.

TABLE 1

| Characteristic | 'NATPHI01' | 'Birkin' |
|---|---|---|
| General coloration of | Dark greyed-red. | Yellow-green. |
| the juvenile petiole. | | |
| General coloration of | Pink. | A mixture of white and |
| the juvenile veins on | | yellow-green. |
| the adaxial foliar | | |
| surface. | | |
| General coloration of | Darker green, relative to | Lighter green, relative to |
| the mature adaxial | ‘Birkin'. | 'NATPHI01'. |
| foliar surface. | | |
| General coloration of | A combination of yellow- | A combination of white, |
| the mature main vein | green and pink. | yellow-green, and green- |
| on the adaxial foliar | | white. |
| surface. | | |

That which is claimed is:

1. A new and distinct variety of Philodendron plant named ‘NATPH101’, substantially as described and illustrated herein.
